(19) United States
(12) Patent Application Publication
(10) Pub. No.: US 2021/0169873 A1
(43) Pub. Date: Jun. 10, 2021

(54) Storage-stable ready-to-use injectable formulations of Trabectedin

(71) Applicant: RK Pharma Solutions LLC, Piscataway, NJ (US)

(72) Inventors: Ravishanker Kovi, Monroe Township, NJ (US); Jayaraman Kannappan, Vadodara (IN); Swaminathan Ramamurthy, Vadodara (IN); Baskaran Ayyanar, Vadodara (IN); Ramesh Pawar, Vadodara (IN); Gaurav Yadav, Jaunpur (US); Ronak Patel, Vadodara (IN); Dixit Panchal, Vadodara (US); Rhagu Kasu, Monmouth Jct., NJ (US); Bhavesh Panchal, Vadodara (IN)

(21) Appl. No.: 17/075,736

(22) Filed: Oct. 21, 2020

(30) Foreign Application Priority Data
Oct. 21, 2019 (IN) 201921042699

(51) Int. Cl. A61K 31/4748; A61K 9/08; A61K 47/10; A61K 47/02; A61K 47/18; A61K 9/00

(52) U.S. Cl. CPC A61K 31/4748; A61K 9/08; A61K 9/0019; A61K 47/02; A61K 47/183; A61K 47/10

(57) ABSTRACT

Ready-to-use liquid parenteral formulations are provided that include trabectedin, at least one of a pharmaceutically acceptable solvent, and at least one pharmaceutically acceptable excipient or adjuvant.

### BACKGROUND

The present application relates to a stable, ready to use, injectable Trabectedin (TBTN) formulation.

Trabectedin, also known as Ecteinascidin 743 or ET-743 (trade name Yondelis®) is an antitumor chemotherapy drug sold by Pharma Mar S.A. and Janssen Products LP (a subsidiary of Johnson and Johnson) under the brand name Yondelis® in US. It is also approved for use in Europe, Russia, and South Korea for the treatment of advanced soft-tissue sarcoma and ovarian cancer. Trabectedin is also in phase II trials for prostate, breast, and paediatric cancers. Trabectedin has the molecular formula C₃₉H₄₃N₃O₁₁S and a molecular weight of 761.84 g/mol. Trabectedin is hydrophobic and has a low solubility in water.

The chemical name of Trabectedin is (1′R,6R,6aR,7R,13S,14S,16R)-5-(acetyloxy)-3′,4′,6,6a,7,13,14,16-octahydro-6′,8,14-trihydroxy-7′,9-dimethoxy-4,10,23-trimethyl-spiro[6,16-(epithiopropanoxymethano)-7,13-imino-12H-1,3-dioxolo[7,8]isoquino[3,2-b][3]benzazocine-20,1′(2′H)-isoquinolin]-19-one. The structural formula is depicted in Table A.

TABLE A

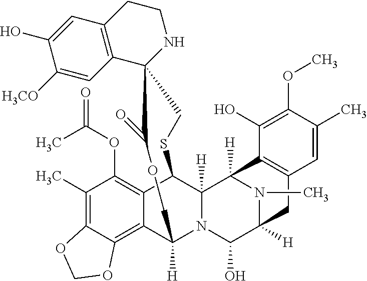 [SMILES: COc1cc2c(cc1O)CCN[C@]21CS[C@@H]2c3c(OC(C)=O)c(C)c4c(c3[C@@H](COC1=O)N1[C@@H]2[C@H]2c3c(cc(C)c(OC)c3O)C[C@@H]([C@@H]1O)N2C)OCO4]

Yondelis® (trabectedin) for injection is supplied as a sterile lyophilized white to off-white powder/cake in a single-dose vial. Each single-dose vial contains 1 mg of trabectedin, 27.2 mg potassium dihydrogen phosphate, 400 mg sucrose, and phosphoric acid and potassium hydroxide (for pH adjustment to 3.6-4.2).

The currently available dosage form of trabectedin for injection is costly to manufacture, distribute, store and inconvenient to use because it is not in a ready-to-use format. Therefore, an aqueous and ready-to-use trabectedin solution formulation in affordable price is highly desirable.

One or more embodiments disclosed herein overcomes or otherwise ameliorates such drawbacks by reducing manufacturing costs, including, for example, by eliminating the need for lyophilisation, labor, and/or equipment costs. In certain embodiments, such results are achieved by eliminating the need to reconstitute the dry powder, which ultimately reduces pharmacy time and the need for refrigeration.

### SUMMARY

The present application provides a stable, ready-to-use injectable Trabectedin solution, which is preferably easy to administer without need for reconstitution and preferably has a desirable solubility, stability, and safety profile.

In one or more embodiments, there is provided a ready-to-use liquid parenteral formulation of Trabectedin.

In one or more further embodiments, there is provided a storage-stable, ready-to-use, injectable liquid parenteral composition including Trabectedin and one or more pharmaceutically acceptable solvents, co-solvents, and/or solubilizing agents.

In still further embodiments, provided are ready-to-use liquid parenteral formulations including Trabectedin, one or more pharmaceutically acceptable solvents, co-solvents, and/or solubilizing agents and at least one pharmaceutically acceptable excipient or adjuvant.

The storage-stable, ready-to-use, injectable compositions of the present application are useful for the treatment of various types of cancer.

In at least one aspect, a ready-to-use liquid parenteral formulation is provided that includes trabectedin, at least one of a pharmaceutically acceptable solvent, and at least one pharmaceutically acceptable excipient or adjuvant.

In at least one embodiment, the pharmaceutically acceptable excipient or adjuvant comprises a pH adjuster selected from the group consisting of orthophosphoric acid and potassium hydroxide.

In at least one embodiment, the formulation has a concentration of trabectedin of less than 10 mg/ml.

In at least one embodiment, the formulation has a concentration of Trabectedin of less than 7 mg/ml.

In at least one embodiment, the formulation has a concentration of Trabectedin of less than 5 mg/ml.

In at least one embodiment, the formulation has a concentration of Trabectedin of less than 3 mg/ml.

In at least one embodiment, the formulation has a concentration of Trabectedin of about 0.5 mg/ml.

In at least one embodiment, the formulation has a concentration of Trabectedin of from about 0.001 w/v to about 0.1 w/v.

In at least one embodiment, the formulation has a concentration of Trabectedin of from about 0.04 w/v to about 0.08 w/v.

In at least one embodiment, the formulation has a concentration of Trabectedin of about 0.05 w/v.

In at least one embodiment, at least 90% purity of the trabectedin is retained after storage for 12 months at 5° C.±3° C.

In at least one embodiment, the formulation has a pH from about 4 to about 9.

In at least one embodiment, the formulation has a pH from about 5 to about 8.

In at least one embodiment, the formulation has a pH from about 6.5 to about 7.3.

In at least one embodiment, the solvent comprises ethanol.

In another aspect, a single dose vial of ready-to-use liquid parenteral formulation is provided that includes trabectedin, ethanol, and sterile water for injection, and wherein the formulation has a pH about 6.5 to about 7.3.

In at least one embodiment, the formulation further includes a pharmaceutically acceptable stabilizing agent.

The In at least one embodiment, the pharmaceutically acceptable stabilizing agent comprises glycine.

In at least one embodiment, the pharmaceutically acceptable stabilizing agent comprises about 125 ml glycine.

In at least one embodiment, 100% purity of the trabectedin is retained after storage for 30 days.

The details of one or more embodiments of the application are set forth in the description below. Other features, objects and advantages of the application will be apparent from the description.

### DETAILED DESCRIPTION OF THE PREFERRED EMBODIMENTS

The present application now will be described more fully hereinafter with reference to the accompanying examples and experiments, in which illustrative embodiments of the application are shown. This application may, however, be embodied in many different forms and should not be construed as limited to the embodiments set forth herein; rather, these embodiments are provided so that this disclosure will be thorough and complete, and will fully convey the scope of the application to those skilled in the art.

The terminology used herein is for the purpose of describing particular embodiments only and is not intended to be limiting of the application. As used herein, the singular forms “a”, “an” and “the” are intended to include the plural forms as well, unless the context clearly indicates otherwise. Unless otherwise defined, all terms (including technical and scientific terms) used herein have the same meaning as commonly understood by one of ordinary skill in the art to which this application belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, “Trabectedin” refers to Trabectedin and the pharmaceutically acceptable salts, solvates, hydrates and anhydrous forms thereof.

As used here in “ready-to-use” when used in connection with a Trabectedin formulation refers to a formulation that includes Trabectedin in dissolved or solubilized form and/or is intended to be used as such or upon further dilution in intravenous diluents.

As used herein, and unless otherwise specified, the term “storage-stable” refers to any Trabectedin-containing composition or formulation having sufficient physical and chemical stability to allow storage at a convenient temperature, such as between about 0° C. and about 50° C., for a commercially reasonable period of time. The phrase “physical stability” refers to maintenance of colour or colourless state, dissolved oxygen level, head space oxygen level and particulate matter, and the phrase “chemical stability” relates to formation of drug-related impurities in terms of total impurities, single maximum individual impurity, or maximum individual unknown impurity. For pharmaceutical products, stability is required for commercially relevant times after manufacturing, such as for about 6, 12, 18, 24, or 36 months, during which time a product is kept in its original packaging under specified storage conditions.

As used herein, and unless otherwise specified, the term “about” means an acceptable error for a particular value as determined by one of ordinary skill in the art, which depends in part on how the value is measured or determined. In certain embodiments, the term about means within 10%, 9%, 8%, 7%, 6%, 5%, 4%, 3%, 2%, 1%, 0.5%, 0.1%, or 0.05% of a given value or range.

In one or more embodiments, ready-to-use liquid parenteral formulations of Trabectedin include Trabectedin and one or more pharmaceutically acceptable solvents, co-solvents, and/or solubilizing agents. In other embodiments, ready-to-use liquid parenteral formulations of Trabectedin include Trabectedin, one or more pharmaceutically acceptable solvents, co-solvents, and/or solubilizing agents, and optionally, one or more pharmaceutically acceptable excipients or adjuvants.

Suitable pharmaceutically acceptable solvents include but are not limited to methanol, ethanol, propanol, butanol and the like. Preferred solvents are methanol and ethanol, more preferentially ethanol.

Pharmaceutically acceptable excipients or adjuvants include but are not limited to one or more preservatives, stabilizing agent, pH adjusting agents, bulking agents, chelating agents and antioxidants.

Examples of pharmaceutically acceptable preservatives include but are not limited to methyl paraben, propyl paraben, benzoic acid, sodium benzoate, sorbic acid, benzethonium chloride, benzalkonium chloride, etc. and any combinations thereof.

Pharmaceutically acceptable stabilizing agent or agents include but are not limited to meglumine, cysteine, methionine, glucose, fructose, mannitol, glycine, sucrose, arginine etc. and combinations thereof.

Examples of pharmaceutically acceptable pH adjusting agents include but are not limited to hydrochloric acid, boric acid, citric acid, acetic acid, orthophosphoric acid, succinic acid, sodium hydroxide, potassium hydroxide, ammonium hydroxide, magnesium oxide, calcium carbonate, magnesium carbonate, magnesium aluminum silicates, malic acid, potassium citrate, sodium phosphate, lactic acid, gluconic acid, tartaric acid, fumaric acid, diethanolamine, monoethanolamine, sodium carbonate, sodium bicarbonate, triethanolamine, etc. and combinations thereof.

Examples of pharmaceutically acceptable bulking agents include but are not limited to mannitol, glycine, meglumine.

Examples of pharmaceutically acceptable chelating agents may include, but are not limited to, citric acid or derivatives thereof, for example, anhydrous citric acid and the like, ethylenediaminetetraacetic acid (EDTA), disodium EDTA or derivatives thereof, or any combination thereof.

Examples of pharmaceutically acceptable anti-oxidants may include, but are not limited to, ascorbic acid, sodium ascorbate, erythorbic acid, potassium metabisulfite, sodium metabisulfite, propionic acid, thiourea, cysteine, n-acetlcysteine, methionine, sodium sulfite, or any combination thereof.

The formulations according to the present application may be in the form of clear injectable solution, suspension or emulsion.

In some embodiments the storage-stable ready-to-use injectable formulation may have a concentration of Trabectedin of less than 10 mg/ml. In other embodiments the injectable formulation may have a concentration of Trabectedin less than 7 mg/ml. In another embodiment the injectable formulation may have a concentration of Trabectedin less than 5 mg/ml. In other embodiments the injectable formulation may have a concentration of Trabectedin of about less than 3 mg/ml. In other embodiments the concentration of Trabectedin in the formulation may be less than about 1 mg/ml. In still other embodiments the concentration of Trabectedin in the formulation may be about 0.05 mg/ml. In still further embodiments the injectable formulation may have a concentration of Trabectedin of from about 0.001 w/v to about 0.1 w/v. In still further embodiments the injectable formulation may have a concentration of Trabectedin of from about 0.04 w/v to about 0.08 w/v. In particular embodiments of the present application, the injectable formulation may have a concentration of Trabectedin of about 0.05% w/v.

The storage-stable, ready-to-use injectable Trabectedin containing formulations disclosed herein do not require any additional reconstitution step at the time of administration.

The formulations have a controlled impurity profile suitable for regulatory approval at various storage conditions. The storage-stable ready-to-use Trabectedin formulations may be stored at 2-8° C. The storage-stable, ready-to-use Trabectedin formulations for injection may retain at least 90% of the potency of Trabectedin after storage for 12 months at 5° C.±3° C.

The storage stable, ready-to-use, injectable formulations may be formulated to provide single or multiple dosage administration. Formulation prepared by following the mentioned process may be further diluted in 500 ml 0.9% w/v Sodium chloride or 5% w/v Dextrose. Also, calculated volume of trabectedin formulation prepared by mentioned process may be further diluted in 500 ml 0.9% w/v Sodium chloride or 5% w/v Dextrose. The single dosage formulation may be packaged in an ampoule, a vial, or a syringe. Multiple dosage formulations may be packaged in a vial. Multiple dosage formulations may preferably include at least one preservative.

The formulations have a pH value from about 4 to about 9. In some embodiments the pH range is from about 5.0 to about 8.0. In other embodiments the pH is about 6.5-7.0.

Storage-stable ready-to-use, injectable formulations disclosed herein contain Trabectedin having a purity of from about 80% to about 120%. In some embodiments the formulation contains Trabectedin having a purity of from about 90% to about 110%. In some embodiments the formulation contains Trabectedin having a purity of about 100%.

Formulations as disclosed herein are useful in the treatment of cancer, including Soft tissue sarcoma and ovarian cancer. Methods of treatment of such cancers are disclosed including administering to an individual in need thereof a therapeutically effective amount of a storage stable, ready-to-use, injectable formulation as disclosed herein.

### EXAMPLES

The following examples are for the illustration only and are not intended in any way to limit the scope of the present application.

### Example 1

TABLE 1

| Ingredients | Qty/vial |
|---|---|
| Trabectedin | 0.5 mg |
| Ethanol | 05 ml |
| Sterile water for injection | 05 ml |
| pH | 6.5-7.3 |
| Orthophosphoric acid (1%) | pH adjustment |
| Potassium hydroxide (0.5%) | pH adjustment |

Referring to Table 1, 05 ml sterile water for injection (pH adjusted to 7.1 using 1% OPA & 0.5% KOH Solution) was taken and 02 ml ethanol was added to the sterile water. Thereafter, 03 ml prep fraction (equivalent to 0.5 mg TBTN) was added to the water/ethanol mixture and the solution was stored at 2-8° C. Stability data is summarized in Table 1A. As can be seen, the purity of the Trabectedin formulation remained at 100% for at least thirty days.

TABLE 1A

| Stability | Input | Day 1 | Day 30 |
|---|---|---|---|
| Purity | 100% | 100% | 100% |

### Example 2

TABLE 2

| Ingredients | Qty./vial |
|---|---|
| Trabectedin | 0.5 mg |
| Ethanol | 05 ml |
| Sterile water for injection | 05 ml |
| Glycine | 125 mg |
| pH | 6.5-7.0 |
| Orthophosphoric acid (1%) | pH adjustment |
| Potassium hydroxide (0.5%) | pH adjustment |

Referring to Table 2, 05 ml glycine solution (125 mg glycine dissolved in 05 ml sterile water for injection) and pH adjusted to 6.7 using 1% OPA & 0.5% KOH solution was taken and 02 ml ethanol was added to the solution. Thereafter, 03 ml prep fraction (equivalent to 0.5 mg TBTN) was added to the pH adjusted solution and the solution was stored at 2-8° C. Stability data is summarized in Table 2A. As can be seen here to, the purity remained at 100% at day 30 and impurities were not detectable.

TABLE 2A

| Stability | RRT | API | Day 1 | Day 30 |
|---|---|---|---|---|
| Purity | 1.00 | 100% | 99.94% | 100% |
| Impurity | 1.63 | ND | 0.06% | ND |

### Example 3

TABLE 3

| Ingredients | Quantity (mg/Vial or PFS) |
|---|---|
| Trabectedin | 0.25 mg |
| Ethanol | q.s. 0.75 ml |
| Sterile water for Injection | 4.25 mL |

Referring to Table 3, Trabectedin (0.25 mg) was weighed and dissolved in ethanol (0.75 ml, previously purged with nitrogen) and mixed gently until a clear solution is formed. The solution was filtered using either 0.22μ PVDF or PES filter. Filtered solution was filled into vial/prefilled syringe (PFS). The solution was stored at room temperature. Stability data is summarized in Table 3A.

TABLE 3A

| | | 25° C./60% | 25° C./60% |
|---|---|---|---|
| | Initial | RH, 14 day | RH, 1M |
| TBTN | 99.37 | 99.50 | 99.07 |
| Deacetyl Imp (0.77 RRT) | 0.09 | 0.05 | 0.02 |
| Dehydroxy Imp (1.06 RRT) | 0.08 | 0.05 | 0.08 |
| TBTN DMME (1.44 RRT) | 0.06 | 0.02 | — |
| Max single unknown Imp | 0.10 | 0.16 | 0.23 |
| Total Imp | 0.33 | 0.28 | 0.37 |

### Example 4

TABLE 4

| Ingredients | Quantity (mg/Vial or PFS) |
|---|---|
| Trabectedin | 1 mg |
| Ethanol | q.s. 3 ml |
| Sterile water for Injection | 17 mL |

Referring to Table 4, Trabectedin (1 mg) was weighed and dissolved in ethanol (3 ml, previously purged with nitrogen) and mixed gently until a clear solution is formed. The solution was filtered using either 0.22μ PVDF or PES filter. Filtered solution was filled into vial/prefilled syringe. The solution was stored at room temperature. Stability data is summarized in Table 4A.

TABLE 4A

| | | 25° C./60% | 25° C./60% |
|---|---|---|---|
| | Initial | RH, 14 day | RH, 1M |
| TBTN | 99.37 | 99.42 | 99.04 |
| Deacetyl Imp (0.77 RRT) | 0.09 | 0.05 | 0.01 |
| Dehydroxy Imp (1.06 RRT) | 0.08 | 0.05 | 0.08 |
| TBTN DMME (1.44 RRT) | 0.06 | 0.02 | — |
| Max single unknown Imp | 0.10 | 0.15 | 0.24 |
| Total Imp | 0.33 | 0.27 | 0.45 |

### Example 5

TABLE 5

| Ingredients | Quantity (mg/Vial or PFS) |
|---|---|
| Trabectedin | 0.25 mg |
| Ethanol | q.s. 0.35 ml |
| Sterile water for Injection | 4.65 ml |

Referring to Table 5, Trabectedin (0.25 mg) was weighed and dissolved in ethanol (0.35 ml, previously purged with nitrogen) and mixed gently until a clear solution is formed. The solution was filtered using either 0.22μ PVDF or PES filter. Filtered solution was filled into vial/prefilled syringe. The solution was stored at room temperature. Stability data is summarized in Table 5A.

TABLE 5A

| | | 25° C./60% | 25° C./60% |
|---|---|---|---|
| | Initial | RH, 14 day | RH, 1M |
| TBTN | 99.74 | 99.66 | 99.39 |
| Deacetyl Imp (0.77 RRT) | 0.06 | 0.26 | 0.27 |
| Dehydroxy Imp (1.06 RRT) | 0.02 | 0.08 | 0.01 |
| TBTN DMME (1.44 RRT) | | ND | ND |
| Max single unknown Imp | 0.09 | ND | 0.15 |
| Total Imp | 0.17 | 0.34 | 0.61 |

### Example 6

TABLE 6

| Ingredients | Quantity (mg/Vial or PFS) |
|---|---|
| Trabectedin | 1 mg |
| Ethanol | q.s. 1.4 ml |
| Sterile water for Injection | 18.6 ml |

Referring to Table 6, Trabectedin (1 mg) was weighed and dissolved in ethanol (1.4 ml, previously purged with nitrogen) and mixed gently until a clear solution is formed. The solution was filtered using either 0.22μ PVDF or PES filter. Filtered solution was filled into vial/prefilled syringe. The solution was stored at room temperature. Stability data is summarized in Table 6A.

TABLE 6A

| | | 25° C./60% | 25° C./60% |
|---|---|---|---|
| | Initial | RH, 14 day | RH, 1M |
| TBTN | 99.74 | 99.88 | 99.24 |
| Deacetyl Imp (0.77 RRT) | 0.06 | 0.06 | 0.26 |
| Dehydroxy Imp (1.06 RRT) | 0.02 | 0.08 | 0.01 |
| TBTN DMME (1.44 RRT) | | ND | ND |
| Max single unknown Imp | 0.09 | ND | 0.16 |
| Total Imp | 0.17 | 0.14 | 0.76 |

What is claimed is:

1. A ready-to-use liquid parenteral formulation, comprising trabectedin, at least one of a pharmaceutically acceptable solvent, and at least one pharmaceutically acceptable excipient or adjuvant.

2. The formulation of claim 1, wherein the pharmaceutically acceptable excipient or adjuvant comprises a pH adjuster selected from the group consisting of Orthophosphoric acid and Potassium hydroxide.

3. The formulation of claim 1, having a concentration of trabectedin of less than 10 mg/ml.

4. The formulation of claim 1, having a concentration of Trabectedin of less than 7 mg/ml.

5. The formulation of claim 1, having a concentration of Trabectedin of less than 5 mg/ml.

6. The formulation of claim 1, having a concentration of Trabectedin of less than 3 mg/ml.

7. The formulation of claim 1, having a concentration of Trabectedin of about 0.5 mg/ml.

8. The formulation of claim 1, having a concentration of Trabectedin of from about 0.001 w/v to about 0.1 w/v.

9. The formulation of claim 1, having a concentration of Trabectedin of from about 0.04 w/v to about 0.08 w/v.

10. The formulation of claim 1, having a concentration of Trabectedin of about 0.05 w/v.

11. The formulation of claim 1, wherein at least 90% purity of the trabectedin is retained after storage for 12 months at 5° C.±3° C.

12. The formulation of claim 1, having a pH from about 4 to about 9.

13. The formulation of claim 1, having a pH from about 5 to about 8.

14. The formulation of claim 1, having a pH from about 6.5 to about 7.3.

15. The formulation of claim 1, wherein the solvent comprises ethanol.

16. A single dose vial of ready-to-use liquid parenteral formulation, comprising trabectedin, ethanol, sterile water for injection, wherein the formulation has a pH about 6.5 to about 7.3.

17. The formulation of claim 16, comprising a pharmaceutically acceptable stabilizing agent.

18. The formulation of claim 17, wherein the pharmaceutically acceptable stabilizing agent comprises glycine.

19. The formulation of claim 17, wherein the pharmaceutically acceptable stabilizing agent comprises about 125 ml glycine.

20. The formulation of claim 16, wherein 100% purity of the trabectedin is retained after storage for 30 days.
